(19) United States
(12) Patent Application Publication
(10) Pub. No.: US 2019/0001136 A1
(43) Pub. Date: Jan. 3, 2019

(54) Implantable Medical Device with a Stylet Channel

(71) Applicant: Boston Scientific Neuromodulation Corporation, Valencia, CA (US)

(72) Inventors: Joshua D. Howard, Sacramento, CA (US); Anne M. Pianca, Santa Monica, CA (US); Benjamin P. Hahn, Stevenson Ranch, CA (US)

(21) Appl. No.: 15/947,381

(22) Filed: Apr. 6, 2018

Related U.S. Application Data
(60) Provisional application No. 62/526,887, filed on Jun. 29, 2017.

(51) Int. Cl. A61N 1/36; A61N 1/05

(52) U.S. Cl. CPC A61N 1/36135; A61N 1/36146; A61N 1/36062; A61N 1/37211; A61N 1/0551; A61N 1/36071; A61N 1/36132

(57) ABSTRACT

An implantable stimulator device is disclosed preferably having a lead portion and an electronics module integrated and implantable as a single unit, which can enable trial stimulation to occur in a fully implanted solution for a lengthened or unlimited duration. The side of the housing of the electronic module includes a stylet channel which proceeds through the housing at an angle to the implantable stimulator's long axis and bends to proceed through the lead portion along the long axis. The stylet channel can receive a lead stylet to allow the lead portion and the electronics module to be properly positioned within the patient. Because the proximal end of the lead stylet exits the housing of the electronics module at an angle, it provides a handle to steer the lead portion for proper placement within the spinal column.

### CROSS REFERENCE TO RELATED APPLICATIONS

This is a non-provisional application of U.S. Provisional Patent Application Ser. No. 62/526,887, filed Jun. 29, 2017, to which priority is claimed, and which is incorporated by reference in its entirety.

### FIELD OF THE TECHNOLOGY

The present application relates to an implantable pulse generator (IPG) with a stylet channel to assist in implantation of the IPG.

### INTRODUCTION

Implantable stimulation devices deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and Deep Brain Stimulators (DBS) to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any Implantable Pulse Generator (IPG) or in any Implantable Medical Device (IMD).

As shown in FIG. 1, a traditional SCS system includes an Implantable Pulse Generator (IPG) 10, which includes a biocompatible device case 12 formed of titanium for example. The case 12 typically holds the circuitry and battery 14 necessary for the IPG 10 to function, which battery 14 may be either rechargeable or primary (non-rechargeable) in nature. The IPG 10 is coupled to electrodes 16 via one or more electrode leads 18 (two of which are shown). The proximal ends of the leads 18 include electrode terminals 20 that are coupled to the IPG 10 at one or more connector blocks 22 fixed in a header 24, which can comprise an epoxy for example. Contacts in the connector blocks 22 make electrical contact with the electrode terminals 20, and communicate with the circuitry inside the case 12 via feedthrough pins 26 passing through a hermetic feedthrough 28 to allow such circuitry to provide stimulation to or monitor the various electrodes 16. In the illustrated system, there are sixteen electrodes 16 split between two leads 18, although the number of leads and electrodes is application specific and therefore can vary. In a traditional SCS application, two electrode leads 18 are typically implanted on the right and left side of the dura within the patient's spinal column.

The electrical stimulation that the IPG 10 is capable of delivering is highly customizable, and various stimulation parameters—including the selected electrodes, electrode current amplitude and polarity, pulse duration, pulse frequency, etc.—can be adjusted. Due to uncertainties in the location of electrodes with respect to neural targets, the physiological response of a patient to stimulation patterns, and the nature of the electrical environment within which the electrodes are positioned, it is challenging to determine the stimulation parameters that might provide effective stimulation therapy for a particular patient. Thus, to determine whether the IPG 10 is capable of delivering effective therapy, and, if so, the stimulation parameters that define such effective therapy, the patient's response to different stimulation parameters is typically evaluated during a trial stimulation phase prior to the permanent implantation of the IPG 10.

As shown in FIG. 2, during the trial stimulation phase, the distal ends of trial leads 18′ are implanted within the epidural space 32 along the spinal column 30. Implantation of the trial leads 18′ (two are shown in FIG. 2) is a relatively simple procedure in which the patient is usually under only light sedation. A local anesthetic is typically administered at the lead insertion site (e.g., in the lower back region), and a needle (e.g., a 14 or 16 gauge needle; not shown) is inserted to create a percutaneous opening 34 through the skin 5. The needle is advanced into the epidural space 32 to the desired lead location under fluoroscopic guidance. The trial lead 18′ is then inserted through the needle using a lead stylet, which acts to stiffen the trial lead 18′ such that it can be maneuvered to the desired location. When the trial lead 18′ is in the desired position (as verified by fluoroscopy), the lead stylet is withdrawn and the process is repeated for any additional trial leads 18′.

During the trial stimulation phase, the proximal ends of the trial leads 18′ having electrode terminals 20 similar to those previously discussed are ultimately coupled to an external trial stimulator (ETS) 40, which as its name implies is external to (i.e., not implanted in) the patient. An external cable box assembly 42 is used to facilitate the connection between the trial leads 18′ and the ETS 40. Each external cable box assembly 42 includes an external cable box 44 (which has a receptacle similar to connector block 22 for receiving the lead), a trial stimulation cable 46, and a male connector 48, which is plugged into a port 50 of the ETS 40. Once connected to the trial leads 18′, the ETS 40 can then be affixed to the patient in a convenient fashion for the duration of the trial stimulation phase, such as by placing the ETS 40 into a belt worn by the patient (not shown). Although not shown in FIG. 2, lead extenders may connect between the trial leads 18′ and the external cable box 44 of the ETS 40.

The ETS 40 essentially mimics operation of the IPG 10 to provide stimulation to the implanted electrodes 16. This allows the effectiveness of stimulation therapy to be verified for the patient, such as whether therapy has alleviated the patient's symptoms (e.g., pain). Trial stimulation using the ETS 40 further allows for the determination of stimulation parameters that can be programmed into the IPG 10 once it is later implanted into the patient. Although not shown, the ETS 40 typically contains a battery within its housing along with stimulation and communication circuitry.

The stimulation parameters executed by the ETS 40 can be provided or adjusted via a wired or wireless link 62 (wireless shown) from a clinician programmer 60. As shown, the clinician programmer 60 comprises a computer-type device, and may communicate wirelessly via link 62 using a communication head (“wand”) 64 wired to the computer. Communication on link 62 may comprise magnetic-inductive or short-range RF telemetry communication standards such as Bluetooth, WiFi, Zibgee, MICS, and the like, and in this regard the ETS 40 and the clinician's programmer 60 and/or communication head 64 may include antennas compliant with the telemetry means chosen. Clinician programmer 60 may be as described in U.S. Patent Application Publication No. 2015/0360038. A hand-held, portable patient external controller 70 may also communicate with the ETS 40 to allow the patient means for providing or adjusting the ETS 70's stimulation parameters, as described in U.S. Patent Application Publication 2015/0080982 for example.

At the end of the trial stimulation phase, the trial leads 18′ are typically explanted and the relatively small percutaneous opening(s) 34 are closed. If trial stimulation proved ineffective for the patient, no further procedures are performed.

By contrast, if stimulation therapy proved effective, IPG 10 can be permanently implanted in the patient, which is often performed in a subsequent procedure after the trial leads 18′ have been explanted. (“Permanent” in this context generally refers to the useful life of the IPG 10). Permanent implantation involves implanting permanent lead(s) 18 (FIG. 1) using the same technique as described above, creating a surgical pocket (e.g., in the buttocks) in which the IPG 10 is positioned, subdermally tunneling the proximal ends of the leads 18 to the pocket, and coupling the leads 18 to the connector blocks 22 in the IPG's header 24. The result is a fully-implanted stimulation therapy solution. The IPG 10 can be programmed with the stimulation parameters that were found to be effective during the trial stimulation phase using the clinician programmer 60 or the patient external controller 70, and/or those stimulation parameters can be further adjusted.

### BRIEF DESCRIPTION OF THE DRAWINGS

FIG. 1 shows an implantable pulse generator (IPG), in accordance with the prior art.

FIG. 2 shows use of trial stimulation preceding implantation of the IPG, including implanted trial leads communicating with an External Trial Stimulator (ETS), in accordance with the prior art.

FIGS. 3A-3C show an improved implantable stimulator device having an electronics module and lead portion that are preferably integrated as a single device, in which the electronics module has an angled stylet channel that exits from the side of the electronic module's housing.

FIGS. 4A-4F show how the implantable stimulator can be implanted, and in particular show how a lead stylet placed in the angled stylet channel can help the implanting physician to position the lead portion of the implantable stimulator at an appropriate location in the spinal column.

### DETAILED DESCRIPTION

While the trial stimulation approach described in the Introduction can be effective, there are certain drawbacks. A first is that because the trial leads 18′ extend through percutaneous opening(s) 34 (FIG. 2) during the trial stimulation phase, there is some risk of infection. While proper bandaging and antibiotics can help mitigate this risk, it is not prudent to continue with the trial stimulation phase for an extended period of time. Therefore, the duration of the trial period is typically limited to about 10 to 14 days for example, a substantial portion of which time the patient is recovering from the trial lead implantation procedure. As a result, there is not much time during which the effectiveness of various stimulation parameters can be evaluated. Even though it may be desirable in some cases to extend the trial stimulation phase, the need to close the opening(s) 34 may cut the experimental period short, thus forcing a premature decision whether to proceed with implantation of the IPG 10.

A further drawback is that the multiple procedures may be required within a short time period. The trial leads 18′ are implanted and then, several days later, the patient undergoes an additional procedure to explant the leads, which can be difficult on the patient. The patient may then additionally need to have a permanent IPG 10 implanted, which implantation can occur at the same time the trial leads 18′ are explanted.

Given these issues, it is desirable to extend the trial stimulation period, or even eliminate the requirement of a multi-step implantation procedure altogether. Traditional external trial stimulation techniques as described earlier are driven at least in part by the size of the IPG 10. Even though manufacturers labor to make IPGs such as 10 as small as possible (e.g., between 10-40 cm³ in volume at the current time), such IPGs are still significant in size, particularly because the battery 14 (whether rechargeable or primary) is relatively large. It is therefore generally desired by patients and clinicians alike that the IPG 10 only be implanted once stimulation therapy effectiveness has been verified during the ETS trial period. But as mentioned, due to the limited time that the percutaneous opening(s) 34 can prudently remain, trial stimulation enables evaluation over a relatively short time period.

Accordingly, the inventors disclose a stimulator system that allows for trial stimulation to occur in a fully implanted solution (i.e., a solution that does not require trial leads 18′ to pass outside of the body through opening(s) such as 34) for a lengthened, and potentially unlimited, duration. The implantable stimulator has a lead portion and an electronics module that are preferably integrated and implanted as a single unit. The electronics module is small compared to the case of conventional Implantable Pulse Generators, but is preferably still large enough to include a battery. To assist with implantation of the implantable stimulator, the side of the housing of the electronic module includes a stylet channel which proceeds through the housing at an angle to the implantable stimulator's long axis. The stylet channel bends at the proximal end of the lead portion and proceeds along the long axis to the distal end of the lead portion. The stylet channel can receive a lead stylet that is sufficiently stiff to allow the lead portion and the electronics module to be properly positioned within the patient. Because the proximal end of the lead stylet will exit the housing of the electronics module at an angle, it provides a handle that can be used to steer the lead portion for proper placement within the patient's spinal column.

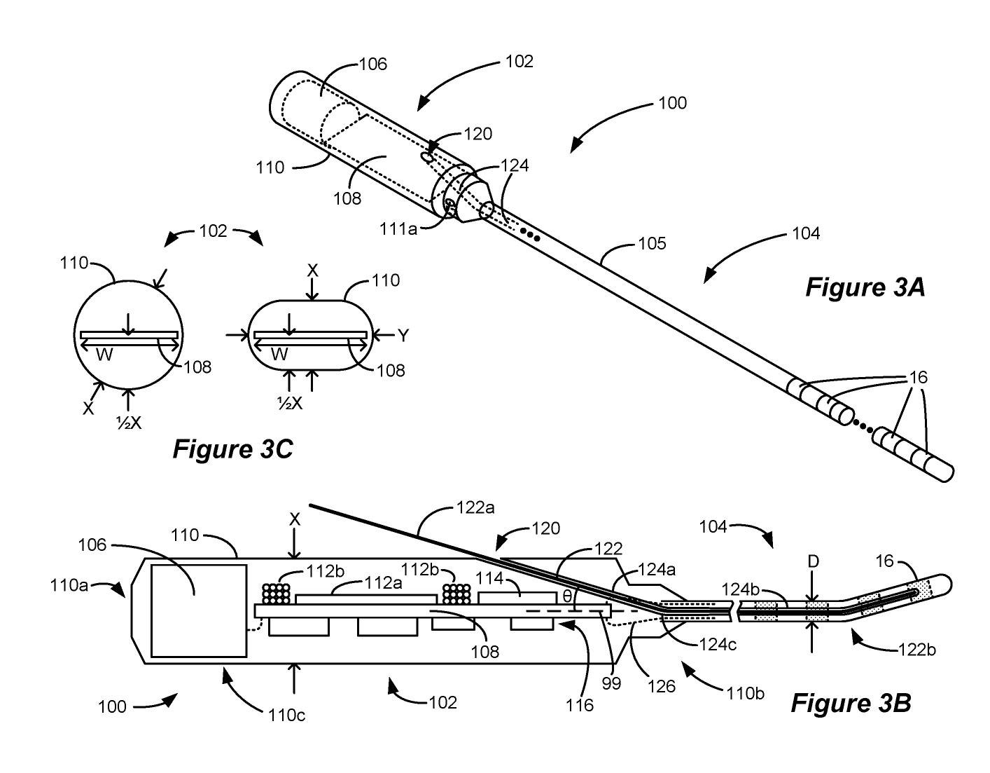

An improved implantable stimulator device 100 as just briefly described is shown in FIGS. 3A through 3C. The implantable stimulator 100 includes an electronics module 102 and a lead portion 104. The lead portion 104 is similar to a traditional electrode lead (e.g., leads 18 or 18′ discussed earlier) and has a lead body 105 along which a number of electrodes 16 are positioned. The lead body 105 is formed of a biocompatible, non-conducting, and preferably flexible material such as, for example, a polymeric material like silicone, polyurethane, polyurea, polyurethane-urea, polyethylene, or the like. The electrodes 16 may be formed from a metal, alloy, conductive oxide, or any other suitable conductive biocompatible material such as platinum, platinum iridium alloy, iridium, titanium, tungsten, palladium, palladium rhodium, or the like. Electrodes 16 may also provide directional stimulation, and thus may comprise well-known split-ring electrodes. In the example shown, implantable stimulator 100 includes only a single lead body which is cylindrical in shape (of diameter D), although this isn't strictly necessary. Other numbers of lead bodies or lead types (e.g., paddle leads) can be used as well.

The electronics module 102 as shown includes a battery 106 and a circuit board 108 contained within a housing 110. Housing 110 can comprise any well-known biocompatible material such as titanium, ceramics, plastics, epoxy or the like. In FIGS. 3A and 3B, the housing 110 is cylindrical, with an outer diameter, X, although the housing 110 may also comprise at least a cylindrical portion. However, the housing 110 may also have a flat shape that is more easily implanted in a patient without bulging through the patient's skin, such as is shown in the right cross section of FIG. 3C, which is discussed further below. For example, the housing 110 may comprises at least a flat portion, which flat portion may comprise one of first and second parallel major sides of the housing as in FIG. 3C. Housing 110 has a distal end 110b to which the lead portion 104 is coupled, an opposing proximal end 110a, and one or more sides 110c therebetween, which side(s) 110c are preferably parallel to a long axis 99 to which the lead portion 104 is parallel. The long axis 99 of the implantable stimulator device 100 is shown along a center of the lead portion 104 and a center of the electronics module 102 in the depicted example, but this is not required of the device's long axis 99 as a general matter. Ends 110a and 110b may not be planar; for example distal end 110b as shown may be generally tapered down to the diameter D of the lead portion 104. Although not illustrated, housing 110 preferably has rounded edges, which renders the implantable stimulator 100 more comfortable when implanted in a patient's tissue.

Circuit board 108 can be coupled to one or more antennas for data communication and/or power receipt. For example, as shown in FIG. 3B, a short-range RF antenna 112a can be included to allow for bi-directional data communications with an external device, such as the clinician programmer 60 or patient controller 70 described earlier (FIG. 2). Such a data antenna 112a can allow the implantable stimulator 100 to receive new or updated stimulation parameters as described earlier, or to report various status data to the external device. Short-range RF antenna 112a can comprise typical configurations, such as a wire, slot, or patch, and can communicate in accordance with communication standards such as those mentioned earlier (e.g., Bluetooth™). Data antenna may also comprise a coil antenna 112b capable of communicating with an external device by magnetic induction. Coil antenna 112b may also be used to wirelessly receive power by magnetic induction from an external device, such as an external charger (not shown). Such wirelessly received power may be rectified and used to recharge battery 106 as is well known. Alternatively, power may be continually wirelessly received at coil antenna 112b from an external device, such as a power patch, see, e.g., U.S. Patent Application Publication 2016/0367822, in which case a battery 106 may not be necessary. Battery 106 may comprise a non-rechargeable primary battery 106, and thus coil antenna 112b may not be necessary, or may be used exclusively for bi-directional data communications.

Circuit board 108 also includes various circuitry to enable stimulation functionality in the implantable stimulator 100. For example, the circuit board 108 may include one or more Application Specification Integrated Circuits (ASICs) including stimulation circuitry for forming stimulation pulses at the electrodes 16 in accordance with programmed stimulation parameters, telemetry circuitry for modulating/demodulating transmitted/received data, etc. Circuit board 108 may include further circuitry 116 such as a microcontroller for organizing operations and for programming the ASIC(s), DC-blocking capacitors which are placed in series with each of the electrode outputs from the ASIC(s), clock circuitry, etc. See, e.g., U.S. Patent Application Publications 2016/0082260, 2012/0095529, and 2018/0071520. Circuit board 108 also includes contact points for the electrode lead wires 126 that proceed down the lead portion 104 and connect to each of the electrodes 16.

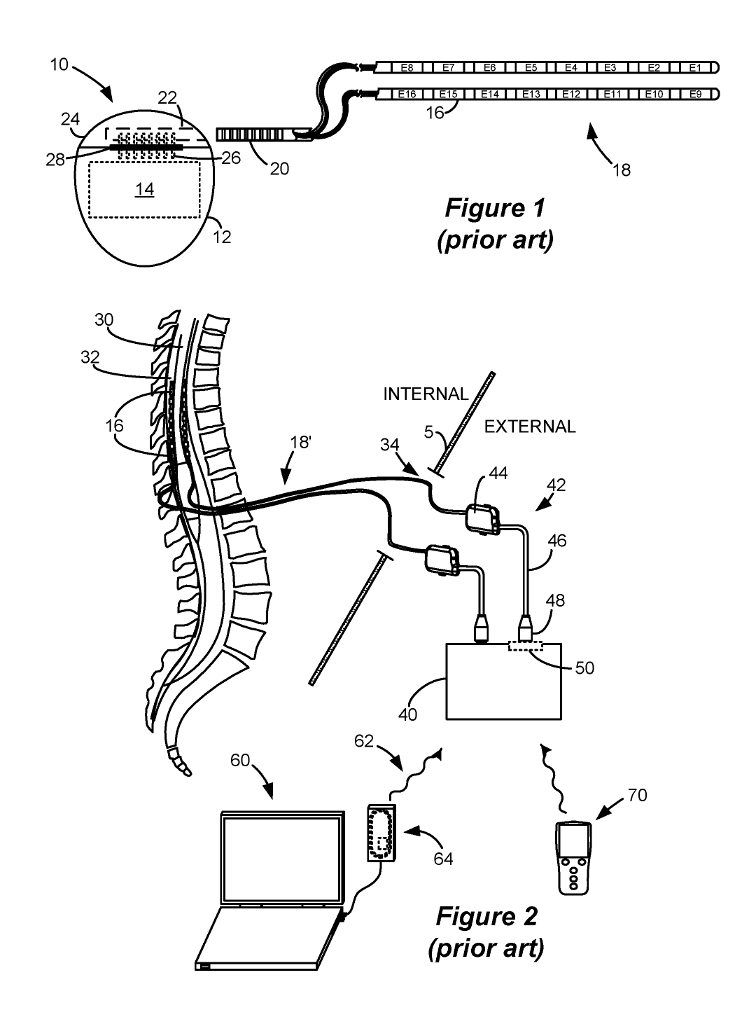

The electronics module 102 and lead portion 104 are preferably integrated together as a single implantable structure, and this can occur in a number of different ways. For example, the lead portion 104 can be attached to the electronics module 102 during assembly, such as by connecting the lead wires 126 to the circuit board 108 and then attaching the lead body 105 to the housing 110. Further, once the lead portion 104 is attached to the electronics module 102, an overmold (not shown) such as silicone may be formed over at least a portion of the housing 110 and lead body 105 to strengthen the connection and provide good hermeticity to prevent fluid ingress. Alternatively, the lead body 105 and housing 110 may be formed as a unitary structure, again using silicone for example. While desirable that the electronics module 102 and lead portion 104 be integrated during assembly, they may also comprise separate pieces and connected later, such as by the surgeon during implantation. For example, the proximal end of the lead portion 104 may be insertable into a port (not shown) on the housing 110 of the electronics modules 102 in other designs, similar to the manner in which a lead is connected to a conventional IPG (see FIG. 1). Notice that the housing 110 of the electronics module 102 may include one or more suture holes 111a to allow the electronics module 102 to be fastened to a patient's tissue, as explained further below.

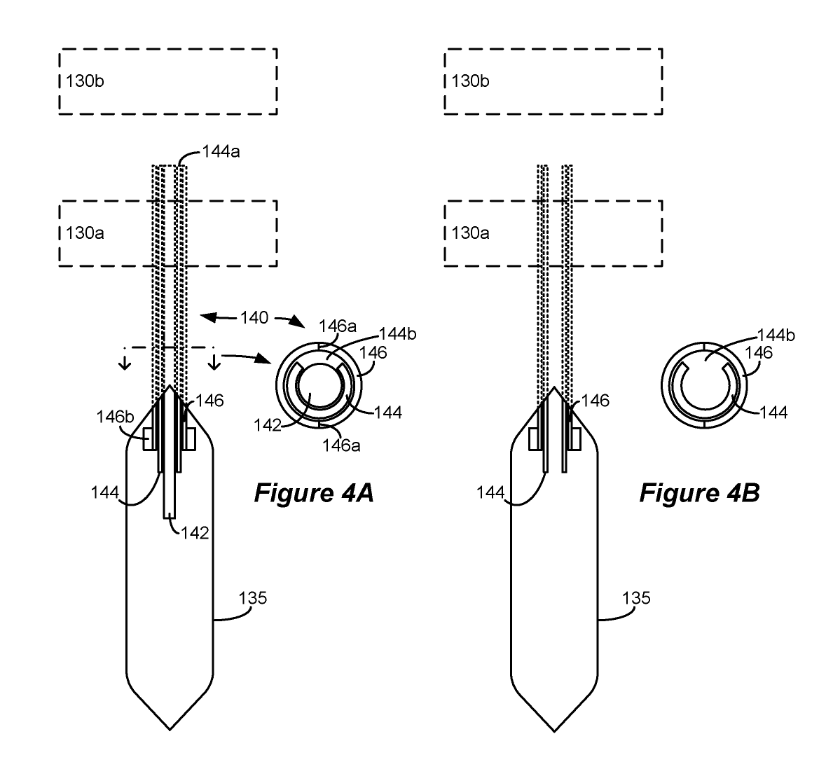

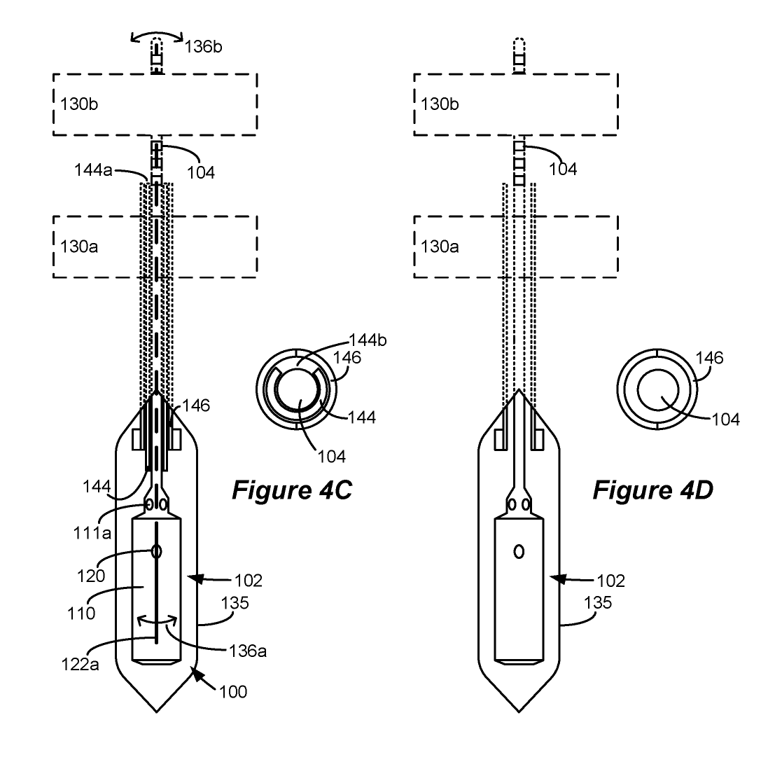

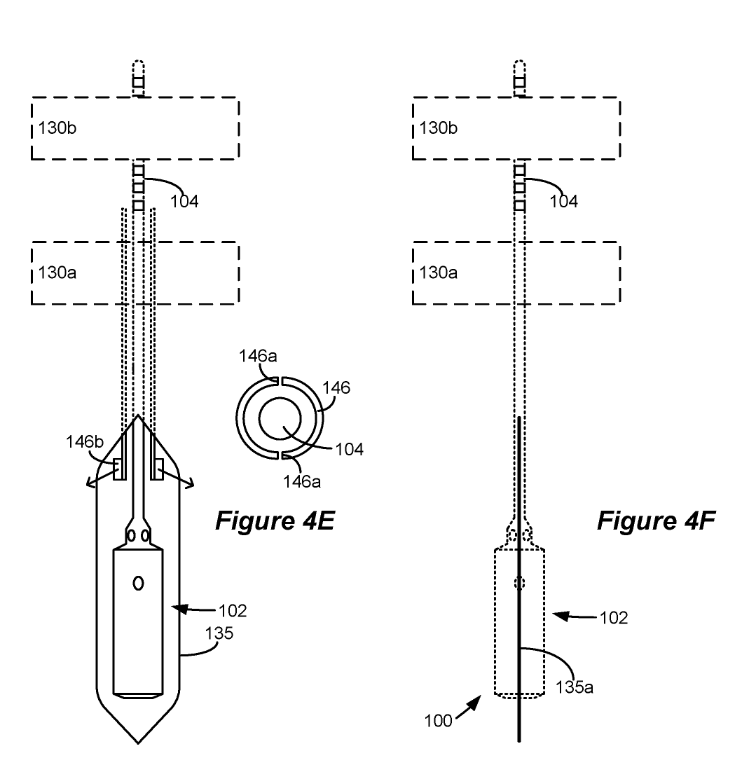

The implantable stimulator 100 includes a stylet channel 124 for receiving a lead stylet 122 (FIG. 3B) that can be used during implantation of the implantable stimulator, as explained further with reference to FIGS. 4A-4F. The stylet channel 124 comprises a portion 124a within the electronic module 102's housing 110, and a portion 124b within the lead portion 104. As shown, the stylet channel portion 124a is angled (0) with respect to the long axis 99 of the implantable stimulator 100, while portion 124b runs parallel to (and preferably along) the long axis 99. (In this regard, and as used herein, structures can be interpreted as “parallel” even if they are collinear with each other). A bend 124c in the stylet channel 124 joins the two portions 124a and 124b. Because the lead stylet 122 is somewhat flexible, the bend 124c in the stylet channel 124 will allow the stylet 122 to pass from the angled portion 124a to the parallel portion 124b, where it come to rest at the distal end of the lead portion 104. Bend 124c and the angled stylet portion 124a are preferably formed within the electronics module housing 110, are preferably rigid, and may be formed of the same material used for the housing, or different materials. By contrast, parallel stylet channel portion 124b is formed in the lead body 105 of the lead portion 104, and therefore like the rest of the lead portion will be generally flexible. Making bend 124c rigid is preferred as this allows the stylet 122 when inserted to be turned and directed straight into the flexible parallel stylet channel portion 124b. Note also that the lead stylet 122 may be slightly bent 122b at its distal end (perhaps after the stylet 122 is inserted through the stylet portions 124a and 124b). This allows the implanting surgeon to better position the distal end of the lead portion 104 in the spinal column, as described further below.

Angled stylet channel portion 124a exits from a side 110c of the housing 110 of the electronics module 102 at an opening 120. Because portion 124a is angled, the proximal end 122a of the lead stylet 122 will likewise exit the opening 120 at an angle, as shown in FIG. 3B. This proximal lead stylet portion 122a is beneficial, as it creates a handle that the implanting surgeon can use to adjust the position of the distal end of the lead portion 104 in the spinal column, as explained below.

There are other benefits to providing an angled stylet channel portion 124a, especially when compared to stylet channels that are straight and proceed through the housing 110 of the electronics module 102 and exit from its proximal end 110a. See, e.g., U.S. Patent Application Publication 2017/0281936 (showing an example of an implantable stimulator with this type of straight stylet channel). A straight stylet channel would proceed through the middle of the housing 110, and as a result components would need to be moved within the housing 110 to accompany the stylet channel. For example, were a straight stylet channel used, the battery 106 could not fully encompass the space within the housing 110 at its proximal end 110a—e.g., its shape could not match that of the housing 110. Instead, use of the angled stylet channel portion 124a at the distal end 110b of the housing 110 allows the battery 106 to match the shape of the housing: if the housing 110 is cylindrical as shown in FIGS. 3A and 3B, the battery 106 may also be cylindrical, with an outer diameter that is just a bit smaller than the inside diameter of the housing. This is important, because the housing 110 of the implantable stimulator 100 is preferably small—for example, 5 cm³ or less—and thus the angled stylet channel portion 124a allows the battery 106 to be made as big as possible at the proximal end 110a.

Likewise, a straight stylet channel would not permit the circuit board 108 and other electronics to be placed at the center inside of the housing 110 as they preferably are in FIGS. 3A-3C. For example, the cross sections of the housing 110 shown in FIG. 3C show that the circuit board 108 may be placed at the center inside the housing 110 where it is widest. When the housing 110 is cylindrical as shown in the left cross section, with diameter X, the circuit board 108 may be placed at ½X, thus maximizing its width, W. If the housing 110 is made flat, comprising upper and lower parallel major sides as in the right cross section, and having a height X and width Y, the circuit board 108 may again be placed at ½X, thus maximizing its width, W. Opening 120, although not shown in FIG. 3C, would preferably be located in one of the upper or lower major side in this example. Increasing circuit board 124 width is desirable, as this increases circuit board area and thus provides more room for the electronics within the housing, such as the antennas 112a and/or 112b, the ASIC(s) 114, and other circuitry 116.

FIGS. 4A-4F show how the implantable stimulator 100 can be implanted in a patient, and further shows how the angled stylet channel portion 124a is helpful in positioning the stimulator. Implantation starts with making an incision to create a pocket 135 in the patient's tissue, which pocket will eventually house the electronics module 102 of the implantable stimulator 100. The location of the pocket 135 can vary, but due to the integrated construction and relatively small size of the implantable stimulator 100, it is preferable that the pocket 135 generally be placed at the patient's physiological midline, such as in the vicinity of the sacrum.

A needle assembly 140 assists in implanting the implantable stimulator 100. The needle assembly 140 can have a nested structure, as shown in the cross section of FIG. 4A. As shown, the needle assembly 140 includes a needle 144 nested within a sheath 146, and a needle stylet 142 nested within the needle 144. A needle assembly 140 of this sort can comprise an Entrada™ needle, Part No. (M365SC42200), manufactured by Boston Scientific Corporation, which is described in a Manual entitled “Boston Scientific: Percutaneous Leads—Directions for Use,” published at https:// www.bostonscientific.com/content/dam/Manuals/us/current-rev-en/91078744-04_RevB_Percutaneous_Leads_DFU_en_US_S.pdf. Note that the needle assembly 140 as depicted in FIG. 4A is not drawn to scale, and does not necessarily include all components of the Entrada needle.

The needle assembly 140 is inserted through the pocket 135 and, as stiffened in particular with the needle stylet 142, is subdermally tunneled to a position where a tip 144a of the needle 144 eventually breaches the epidural space within the spinal column, which in FIG. 4A is shown to occur between vertebrae 130a and 130b. Fluoroscopic guidance is typically used to determine the location of the needle tip 144a during needle assembly 140 insertion. At this point, the needle stylet 142 is pulled out of and removed from the needle assembly 140, as shown in FIG. 4B. Although not shown, but as explained in the above-referenced Manual, a Loss of Resistance (LOR) adaptor may intervene as an extra nested structure between the needle stylet 142 and the needle 144. A needle blank may be inserted though the LOR adaptor under fluoroscopic guidance to verify that the needle has entered the epidural space. Again, these optional structures are not shown.

After the needle stylet 142 (and optional LOR adaptor) has been removed, the implantable stimulator 100 can be introduced, as shown in FIG. 4C. In preparation, the lead stylet 122 is inserted though opening 120 on a side 110c of the housing 110, and routed through the angled portion 124a and the straight portion 124b until its distal end reaches the end of the lead portion 104. As noted earlier, this results in the proximal lead stylet portion 122a exiting the opening 120 at an angle θ as shown in FIG. 3B. The lead portion 104 as stiffened with the lead stylet 122 is then inserted within the needle 144. Using fluoroscopic guidance, the distal end of the lead portion 104 with the electrodes 16 is made to pass through the epidural space beyond the point at which the needle tip 144a had earlier breached the epidural space. Rotating the electronics module 102 around its long axis 99 (FIG. 3B) can be useful to help guide the distal end of the lead portion 104 as it advances through the epidural space, because this will rotate the bent stylet tip 122b (FIG. 3B) to change its trajectory.

At this point of the procedure, the surgeon will typically seek to position the lead portion 104 at a desired location in the epidural space, and such positioning is assisted by virtue of the implantable stimulator 100's design. As discussed earlier, angled stylet channel portion 124a causes the proximal end 122a of the lead stylet 122 to exit the opening 120 at an angle, which would be out of the page in FIG. 4C. This proximal stylet end 122a will thus protrude outside of the pocket 135, providing a handle that the surgeon can use to move the electronics module 102 back and forth, as shown by arrow 136a. This will in turn cause the distal end of the lead portion 104 to move back and forth in the spinal column, as shown by arrows 136b. Note that movement of the electronics module 102 in one direction (e.g., to the left) may cause the lead portion 104 to move in the opposite direction (e.g., to the right), pivoting around the point at which the needle tip 144a breached the epidural space.

Once the lead portion 104 is properly positioned, the needle 144 can be removed, as shown in FIG. 4D. In this regard, notice that the needle 144 is preferably includes a slot 144b along its entire length, which slot 144b preferably faces upwards, i.e., out of the pocket 135 and away from the patient. Thus, when removing the needle 144, the electronics module 102 can be lifted upwards such that the proximal end of the lead portion 104 peels away from the slot 144b while the needle 144 is pulled out of the pocket 135.

Next, as shown in FIG. 4E, the sheath 146 is removed. In this regard, the sheath 146 is preferably formed of a thin biocompatible plastic, and includes two scores 146a along its entire length. These scores 146a are strong enough to keep the needle assembly 140 intact (particularly the slotted needle 144), but which can be ripped apart to separate the sheath 146 into two sections as shown in the cross section. Scores 146a can comprise perforations for example. Two handles 146b are connected to each section of the sheath. The sheath is removed by pulling the handles 146b apart and backwards as shown by the arrows, which rips the sheath 146 at its scores 146a and pulls the separated sheath sections out of the patient, leaving the lead portion 104 of the implantable stimulator 100 in place.

Thereafter, the housing 110 of the electronics module 102 can be sutured at suture holes 111a to the patient's tissue within the pocket 135 (not shown) to keep the electronics module 102 from moving or rotating. Opening 120 in the housing 110 can be plugged if desired to prevent fluid ingress into the stylet channel 124. With the electronics module 102 so placed in the pocket 135, the pocket may then be stitched closed at 135a. At this point the implantable stimulator 100 is fully implanted.

The implantable stimulator 100 may now be activated, and programmed with stimulation parameters to provide stimulation pulses to the patient's spinal column at electrodes 16, as described earlier. In particular, the implantable stimulator 100 may be used during a trial stimulation period, but with the significant benefit that the stimulator is fully implanted without percutaneous openings. This mitigates the risk of infection, and allows trial stimulation to proceed for a longer period of time (e.g., longer than 10-14 days) than occurs when traditional trial stimulation techniques using percutaneous trial lead(s) 18′ are used, as described earlier with reference to FIG. 2.

If implantation of a more traditional IPG 10 is eventually warranted, the procedures to implant the implantable stimulator 100, explant the implantable stimulator 100, and implant the IPG 10 can be spaced further in time, providing the patient more time to heal between procedures. Even if the implantable simulator 100 is providing sufficient stimulation therapy, implantation of a more traditional IPG 10 may be warranted in particular because the IPG 10 likely has a larger battery 14 (FIG. 1) than the battery 106 within the smaller implantable stimulator 100, and thus will operate for a longer period of time (if batteries 14 and 106 are non-rechargeable primary batteries), or require less frequent recharging (if batteries 14 and 106 are rechargeable).

Alternatively, the implantable stimulator 100 if providing effective stimulation therapy may be used indefinitely to provide therapeutic stimulation beyond a trial period when stimulation parameters are being adjusted to find appropriate setting for the patient. By contrast, if stimulation therapy is proving ineffective, the implantable stimulator 100 may be explanted at a time convenient for the patient and clinician.

While the inventions disclosed have been described by means of specific embodiments and applications thereof, numerous modifications and variations could be made by those skilled in the art without departing from the scope of the inventions set forth in the following claims or its equivalents.

What is claimed is:

1. An implantable stimulator device, comprising:
an electronics module having a housing with a side parallel to a long axis of the device, the housing containing stimulation circuitry configured to generate electrical stimulation;
a lead portion coupled to the housing, the lead portion comprising at least one electrode configured to provide the electrical stimulation to a patient's tissue, wherein the lead portion is parallel to the long axis; and
a stylet channel configured to receive a lead stylet that assists with implantation of the implantable stimulator device in a patient, wherein the stylet channel passes through an opening on the side of the housing and through the lead portion.

2. The implantable stimulator device of claim 1, wherein the side of the housing comprises at least a cylindrical portion.

3. The implantable stimulator device of claim 1, wherein the side of the housing comprises at least a flat portion, and wherein the side comprises one of first and second parallel major sides of the housing.

4. The implantable stimulator device of claim 1, further comprising a circuit board contained within the housing, wherein the stimulation circuitry is mounted to the circuit board, and a battery contained within the housing.

5. The implantable stimulator device of claim 4, wherein the battery is adjacent a proximal end of the housing, and wherein the circuit board is adjacent a distal end of the housing, wherein the lead portion is coupled to the distal end of the housing.

6. The implantable stimulator device of claim 1, wherein the electronics module and the lead portion are integrated during manufacturing as a single implantable device.

7. The implantable stimulator device of claim 1, wherein the lead portion is coupled to a port on the housing.

8. The implantable stimulator device of claim 1, further comprising a coil antenna, wherein the coil antenna is configured to wirelessly receive at least one of power or data from an external device.

9. The implantable stimulator device of claim 1, wherein the stylet channel comprises a first portion that is angled with respect to the long axis, and wherein the stylet channel comprises a second portion that is parallel to the long axis through the lead portion.

10. The implantable stimulator device of claim 9, wherein the stylet channel further comprises a bend that joins the first portion and the second portion.

11. An implantable stimulator device, comprising:
an electronics module having a housing, the housing containing within stimulation circuitry configured to generate electrical stimulation;
a lead portion coupled to the housing, the lead portion comprising at least one electrode configured to provide the electrical stimulation to a patient's tissue, wherein the lead portion is parallel to a long axis of the device; and
a stylet channel configured to receive a lead stylet that assists with implantation of the implantable stimulator device in a patient, wherein the stylet channel comprises a first portion and a second portion, wherein the first portion passes through the housing at an angle with respect to the long axis, and wherein the second portion passes through the lead portion parallel to the long axis.

12. The implantable stimulator device of claim 11, wherein the housing comprises a distal end, a proximal end, and a side between the distal and proximal ends, wherein the lead portion is coupled to the distal end of the housing.

13. The implantable stimulator device of claim 12, wherein the first portion exits at an opening on the side of the housing.

14. The implantable stimulator device of claim 12, further comprising a circuit board contained within the housing, wherein the stimulation circuitry is mounted to the circuit board, and a battery contained within the housing.

15. The implantable stimulator device of claim 14, wherein the battery is adjacent the proximal end, and wherein the circuit board is adjacent the distal end.

16. The implantable stimulator device of claim 11, wherein the electronics module and the lead portion are integrated during manufacturing as a single implantable device.

17. The implantable stimulator device of claim 11, wherein the lead portion is coupled to a port on the housing.

18. The implantable stimulator device of claim 11, wherein the long axis is along a center of the lead portion.

19. The implantable stimulator device of claim 11, further comprising a coil antenna, wherein the coil antenna is configured to wirelessly receive at least one of power or data from an external device.

20. The implantable stimulator device of claim 11, wherein the stylet channel further comprises a bend that joins the first portion and the second portion.
